(19) United States
(12) Patent Application Publication
(10) Pub. No.: US 2024/0389913 A1
(43) Pub. Date: Nov. 28, 2024

(54) CATHETER ASSEMBLY AND CATHETER SYSTEM

(71) Applicant: TERUMO KABUSHIKI KAISHA, Tokyo (JP)

(72) Inventor: Shota TSUMAKI, Kai-shi (JP)

(73) Assignee: TERUMO KABUSHIKI KAISHA, Tokyo (JP)

(21) Appl. No.: 18/792,667

(22) Filed: Aug. 2, 2024

Related U.S. Application Data
(63) Continuation of application No. PCT/JP2023/003492, filed on Feb. 3, 2023.

(30) Foreign Application Priority Data
Feb. 9, 2022 (JP) 2022-018693

(51) Int. Cl. A61B 5/15

(52) U.S. Cl. CPC A61B 5/150992; A61B 5/150824

(57) ABSTRACT

A catheter assembly includes: a tubular catheter shaft; and an inner needle inserted into a lumen of the catheter shaft, the inner needle including an insertion distal end surrounded by a catheter distal end which is a distal end of the catheter shaft in an initial state of the catheter assembly in which the inner needle is inserted into the lumen of the catheter shaft. A wavelength conversion member is included in at least one of the catheter distal end or the insertion distal end, the wavelength conversion member including a wavelength conversion material that converts near-infrared light into visible light.

### CROSS-REFERENCE TO RELATED APPLICATIONS

This is a bypass continuation of PCT Application No. PCT/JP2023/003492, filed on Feb. 3, 2023, which claims priority to Japanese Application No. JP2022-018693, filed on Feb. 9, 2022. The entire contents of these applications are incorporated herein by reference.

### BACKGROUND

### Technical Field

The present disclosure relates to a catheter assembly and a catheter system.

### Related Art

The catheter assembly includes, for example, a tubular catheter shaft and an inner needle inserted into a lumen of the catheter shaft. In a blood vessel puncture procedure using such a catheter assembly, a blood vessel is punctured with the inner needle and the catheter shaft in a state where the inner needle protrudes from the distal end of the catheter shaft. In this case, a user confirms the securement of the catheter shaft to the blood vessel by visually observing the backflow (flashback) of blood from a groove or a side hole provided in the inner needle to a space between the inner needle and the catheter shaft.

JP 2015-126885 A discloses a catheter system allowing for confirming a position of a catheter in a blood vessel. The catheter system includes a catheter including a luminescent agent, an irradiation unit, a camera, and a monitor. The irradiation unit irradiates the luminescent agent of the catheter with near-infrared light. The luminescent agent emits near-infrared fluorescence when being irradiated with the near-infrared light. The camera receives the near-infrared fluorescence emitted by the luminescent agent. The monitor displays an image captured by the camera.

### SUMMARY

In the catheter assembly of the related art, because a time lag occurs until the flashback can be visually recognized after the catheter distal end is located in the blood vessel, it is not possible to immediately know that the catheter shaft is secured in the blood vessel. Furthermore, in a case in which the catheter distal end penetrates the back wall of the blood vessel or the catheter distal end comes out of the blood vessel after the catheter shaft is secured in the blood vessel, the user cannot easily recognize that the catheter distal end is no longer located in the blood vessel. The above-described catheter system disclosed in JP 2015-126885 A requires a camera and a monitor to know the position of the catheter, and thus has a problem of being large and complicated.

An object of the present disclosure is to solve the above-described problems.
(1) According to an aspect of the present disclosure, there is provided a catheter assembly including: a tubular catheter shaft; and an inner needle inserted into a lumen of the catheter shaft, in which the inner needle includes an insertion distal end surrounded by a catheter distal end which is a distal end of the catheter shaft in an initial state of the catheter assembly in which the inner needle is inserted into the lumen of the catheter shaft, a wavelength conversion member is provided in at least one of the catheter distal end or the insertion distal end, and the wavelength conversion member includes a wavelength conversion material that converts near-infrared light into visible light.
(2) In the catheter assembly according to (1), it is preferable that the wavelength conversion member is provided in the catheter distal end, and the catheter shaft includes a catheter body that is located on a proximal end side with respect to the catheter distal end and is not capable of converting the near-infrared light into the visible light.
(3) In the catheter assembly according to (2), it is preferable that the wavelength conversion member constitutes a wall portion of at least a part of the catheter distal end.
(4) In the catheter assembly according to (2), it is preferable that the catheter distal end includes a non-wavelength conversion member that does not include the wavelength conversion material.
(5) In the catheter assembly according to (4), it is preferable that the wavelength conversion member and the non-wavelength conversion member are adjacent to each other in a circumferential direction or a radial direction of the catheter shaft.
(6) In the catheter assembly according to (2), it is preferable that the wavelength conversion member is applied on at least one of an outer circumferential surface or an inner circumferential surface of the catheter distal end.
(7) In the catheter assembly according to any one of (1) to (6), it is preferable that the wavelength conversion material converts the near-infrared light having a wavelength of more than 700 nm into the visible light having a wavelength ranging from 360 nm to 700 nm.
(8) In the catheter assembly according to any one of (1) to (7), it is preferable that the wavelength conversion member is provided only in the catheter distal end or the insertion distal end.
(9) According to another aspect of the present disclosure, there is provided a catheter system including: the catheter assembly according to any one of (1) to (8); and an irradiation unit for emitting the near-infrared light.

According to the present disclosure, by irradiating the wavelength conversion member with the near-infrared light in a state before blood vessel access in which the catheter distal end is located in the subcutaneous tissue, the visible light can be generated from the wavelength conversion member. Therefore, the user can know that the catheter distal end is located outside the blood vessel by visually recognizing the visible light. The near-infrared light is absorbed by the blood flowing in the blood vessel. Therefore, when the catheter distal end is located in the blood vessel, the near-infrared light is not emitted to the catheter distal end. That is, the visible light generated from the wavelength conversion member disappears. Therefore, the user can immediately know that the catheter shaft is secured in the blood vessel by visually recognizing that the visible light generated from the wavelength conversion member disappears.

Furthermore, in a case in which the catheter distal end penetrates the back wall of the blood vessel or the catheter distal end comes out of the blood vessel after the catheter shaft is secured in the blood vessel, the wavelength conversion member emits light again. Therefore, the user can easily know that the catheter distal end is not located in the blood vessel by visually recognizing the re-emission from the wavelength conversion member.

Furthermore, by directly visually recognizing the visible light generated from the catheter distal end, it is possible to know that the catheter shaft is secured in the blood vessel without using a camera, a monitor, and the like. Therefore, it is possible to prevent the catheter system from becoming large and complicated.

### BRIEF DESCRIPTION OF DRAWINGS

FIG. 1 is a configuration explanatory diagram of a catheter system according to an embodiment of the present disclosure;

FIG. 2 is an exploded perspective view of a catheter assembly of FIG. 1;

FIG. 3 is a longitudinal cross-sectional view of a distal end of a catheter assembly of FIG. 1;

FIG. 4 is a transverse cross-sectional view of a catheter assembly of FIG. 3 taken along line IV-IV;

FIG. 5A is a first explanatory diagram of a blood vessel puncture procedure using a catheter assembly;

FIG. 5B is an explanatory view illustrating how a puncture target site is seen in FIG. 5A;

FIG. 6A is a second explanatory diagram of a blood vessel puncture procedure using a catheter assembly;

FIG. 6B is an explanatory view illustrating how a puncture target site is seen in FIG. 6A;

FIG. 7A is a third explanatory diagram of a blood vessel puncture procedure using a catheter assembly;

FIG. 7B is an explanatory view illustrating how a puncture target site is seen in FIG. 7A;

FIG. 8A is a transverse cross-sectional view of a catheter assembly according to a first modification example;

FIG. 8B is a transverse cross-sectional view of a catheter assembly according to a second modification example;

FIG. 9A is a longitudinal cross-sectional view of a distal end of a catheter assembly according to a third modification example;

FIG. 9B is a transverse cross-sectional view of a catheter assembly of FIG. 9A taken along line IXB-IXB;

FIG. 10A is a longitudinal cross-sectional view of a distal end of a catheter assembly according to a fourth modification example; and

FIG. 10B is a transverse cross-sectional view of a catheter assembly of FIG. 10A taken along line XB-XB.

### DETAILED DESCRIPTION

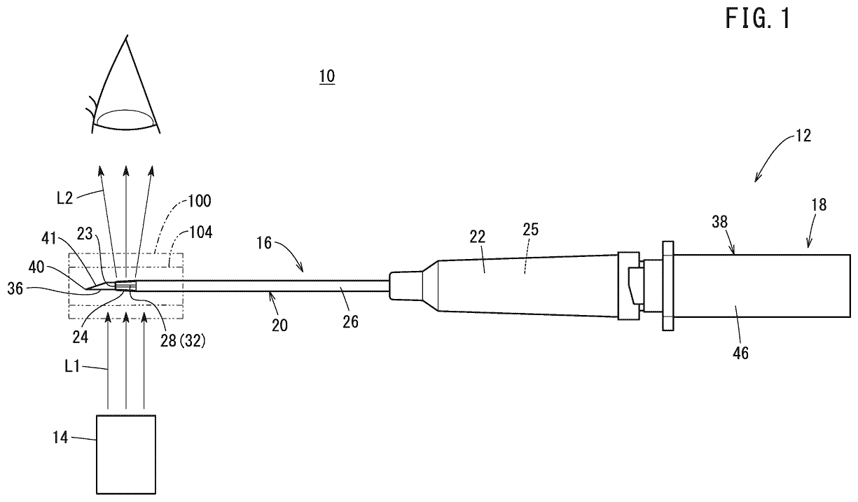

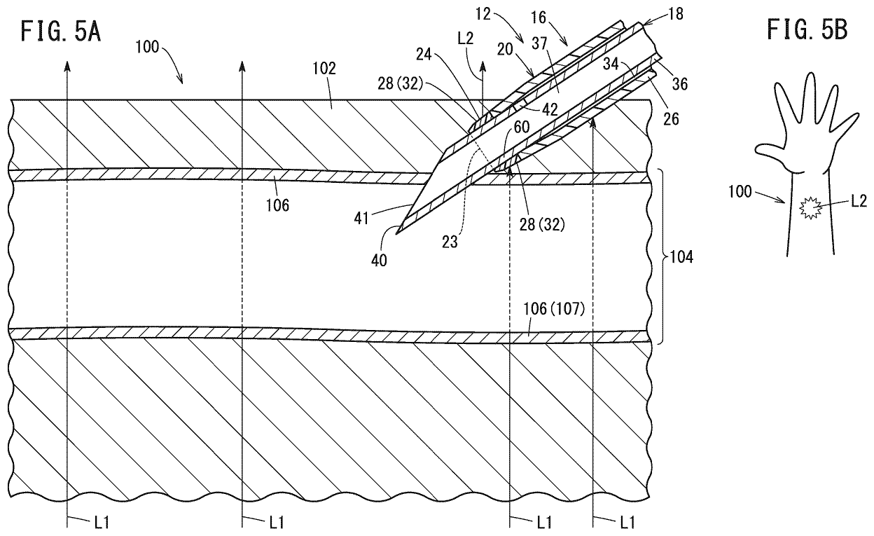

As illustrated in FIG. 1, a catheter system 10 according to an embodiment of the present disclosure includes a catheter assembly 12 and an irradiation unit 14. The catheter assembly 12 is, for example, an indwelling needle for administering an infusion (medicinal solution) into a blood vessel 104 of a puncture target site 100 (see FIG. 5A).

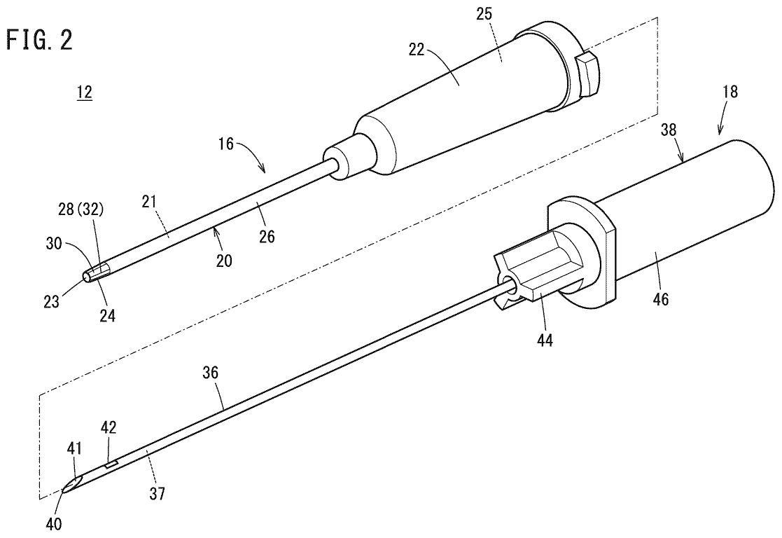

As illustrated in FIGS. 1 and 2, the catheter assembly 12 includes a catheter member 16 and a needle member 18. The catheter member 16 includes a catheter shaft 20 and a catheter hub 22.

The catheter shaft 20 has flexibility. The catheter shaft 20 is a tubular member that can be continuously inserted into the blood vessel 104. The catheter shaft 20 has a lumen 21 extending along an axial direction over the entire length thereof. An inner needle 36 of the needle member 18 is inserted into the lumen 21 of the catheter shaft 20. A distal end opening 23 communicating with the lumen 21 is formed at the distal end of the catheter shaft 20.

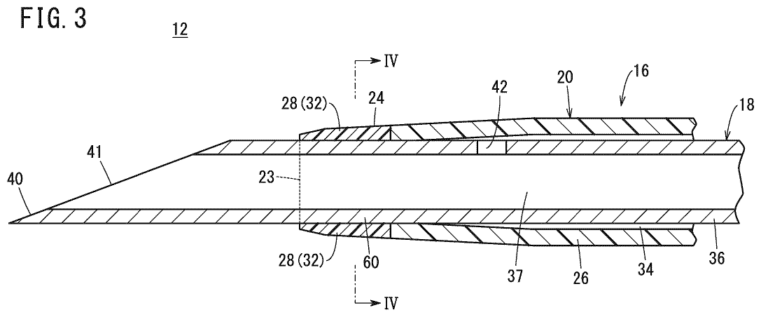

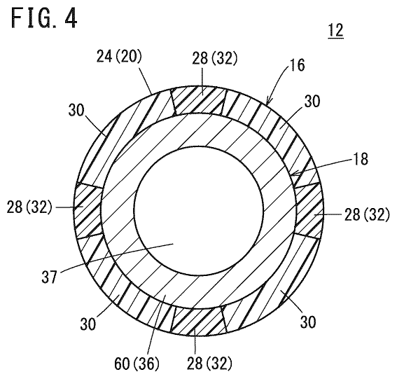

As illustrated in FIGS. 3 and 4, the catheter shaft 20 includes a catheter distal end 24 and a catheter body 26. The catheter distal end 24 constitutes a distal end of the catheter shaft 20. The inner circumferential surface of the catheter distal end 24 is in contact with the outer circumferential surface of the inner needle 36.

In FIG. 4, the catheter distal end 24 includes a plurality of wavelength conversion members 28 and a plurality of non-wavelength conversion members 30. Each of the wavelength conversion members 28 includes a wavelength conversion material 32 (light up-conversion material) that converts near-infrared light L1 into visible light L2. Each of the non-wavelength conversion members 30 cannot convert the near-infrared light L1 into the visible light L2. In other words, the non-wavelength conversion member 30 does not include the wavelength conversion material 32.

The wavelength conversion material 32 converts the near-infrared light L1 into the visible light L2 having a wavelength ranging from 360 nm to 700 nm. The wavelength conversion material 32 includes, for example, an inorganic light up-conversion luminous body or an organic light up-conversion luminous body. The inorganic light up-conversion luminous body includes, for example, a rare earth element. The organic light up-conversion luminous body includes, for example, an organometallic complex or a polycyclic aromatic compound.

Examples of a constituent material of the non-wavelength conversion member 30 include a fluorine-based resin such as polytetrafluoroethylene (PTFE), an ethylene-tetrafluoroethylene copolymer (ETFE), and a perfluoroalkoxy fluorine resin (PFA), an olefin-based resin such as polyethylene and polypropylene or a mixture thereof, polyurethane, polyester, polyamide, a polyether nylon resin, and a mixture of the olefin-based resin and an ethylene-vinyl acetate copolymer.

In the present embodiment, the catheter distal end 24 includes four wavelength conversion members 28 and four non-wavelength conversion members 30. The wavelength conversion member 28 constitutes a wall portion of at least a part of the catheter distal end 24. The catheter distal end 24 is configured by alternately disposing the wavelength conversion members 28 and the non-wavelength conversion members 30 in a circumferential direction of the catheter shaft 20.

The wavelength conversion member 28 and the non-wavelength conversion member 30 are adjacent to each other in the circumferential direction of the catheter shaft 20. A plurality of the wavelength conversion members 28 are disposed at equal intervals (for example, at intervals of) 90° in the circumferential direction of the catheter distal end 24. Each of the wavelength conversion members 28 and each of the non-wavelength conversion members 30 extend over the entire length of the catheter distal end 24 in the axial direction of the catheter shaft 20. The number of the wavelength conversion members 28, and the size and shape of the wavelength conversion member 28 can be set as appropriate.

The wavelength conversion member 28 is made only of the wavelength conversion material 32. However, the wavelength conversion member 28 may be made of a material obtained by kneading the constituent material of the non-wavelength conversion member 30 and the wavelength conversion material 32. In this case, the content of the wavelength conversion material 32 can be appropriately set. The wavelength conversion member 28 is provided only in the catheter distal end 24.

In FIGS. 2 and 3, the catheter body 26 constitutes the entire catheter shaft 20 on the proximal end side with respect to the catheter distal end 24. The catheter body 26 has a substantially constant outer diameter and inner diameter over the entire length. The catheter body 26 is made of a material that cannot change the near-infrared light L1 to the visible light L2. Examples of the constituent material of the catheter body 26 include the same material as the constituent material of the non-wavelength conversion member 30 described above. The catheter body 26 may be made of a material that absorbs the near-infrared light L1.

The catheter body 26 includes a reflection suppressing portion that suppresses reflection of the visible light L2. The reflection suppressing portion is formed by, for example, making the catheter body 26 itself black. Furthermore, the reflection suppressing portion may be formed by applying a black paint to the outer circumferential surface of the catheter body 26.

At least a part of the catheter body 26 may be made of a material having transparency such that blood flowing through a space (blood introduction passage 34) between the catheter body 26 and the inner needle 36 can be visually recognized from the outside of the catheter body 26.

As illustrated in FIG. 2, the catheter hub 22 is attached to the proximal end of the catheter shaft 20. The catheter hub 22 is formed in a hollow shape (cylindrical shape). The outer circumferential surface of the proximal end of the catheter shaft 20 is fixed to the distal end of the catheter hub 22 by appropriate fixing means such as caulking, fusion, or adhesion. The catheter hub 22 has a lumen 25 communicating with the lumen 21 of the catheter shaft 20.

Although not illustrated, a hemostasis valve, a seal member, and a plug are disposed in the lumen 25 of the catheter hub 22. The catheter hub 22 has transparency such that blood flowing into the lumen 25 of the catheter hub 22 can be visually recognized from the outside of the catheter hub 22.

The catheter hub 22 is preferably made of a material harder than the catheter shaft 20. The constituent material of the catheter hub 22 is not particularly limited, but for example, a thermoplastic resin such as polypropylene, polycarbonate, polyamide, polysulfone, polyarylate, a methacrylate-butylene-styrene copolymer, polyurethane, an acrylic resin, or an ABS resin can be suitably used.

As illustrated in FIGS. 2 and 3, the needle member 18 includes the inner needle 36 and a needle hub 38. The inner needle 36 is a tubular member having rigidity, which is capable of puncturing the puncture target site 100. The inner needle 36 includes a lumen 37 extending along the axial direction. The inner needle 36 is inserted into the lumen 21 of the catheter shaft 20 and the lumen 25 of the catheter hub 22 in an initial state (assembled state) of the catheter assembly 12. The inner needle 36 has an insertion distal end 60 surrounded by the catheter distal end 24 in the initial state of the catheter assembly 12 (see FIG. 3).

Examples of the constituent material of the inner needle 36 include metal materials such as stainless steel, aluminum, an aluminum alloy, titanium, and a titanium alloy. The inner needle 36 is formed sufficiently longer than the catheter shaft 20 and protrudes from the distal end opening 23 of the catheter shaft 20 in the initial state of the catheter assembly 12 (see FIGS. 1 and 3). The distal end of the inner needle 36 has a blade surface 40 inclined with respect to the axis of the inner needle 36. An elliptical distal end opening 41 communicating with the lumen 37 of the inner needle 36 is formed on the blade surface 40.

The inner needle 36 has a side hole 42 penetrating from the inner circumferential surface to the outer circumferential surface of the inner needle 36. The side hole 42 guides blood flowing into the lumen 37 of the inner needle 36 to the blood introduction passage 34. In the initial state of the catheter assembly 12, the side hole 42 faces the inner circumferential surface of the catheter body 26. In other words, in the initial state of the catheter assembly 12, the side hole 42 is located on the proximal end side of the inner needle 36 with respect to the catheter distal end 24. Therefore, the blood flowing through the blood introduction passage 34 does not come into contact with the wavelength conversion member 28. The outer surface of the inner needle 36 may have a groove (not illustrated) for guiding the blood to the blood introduction passage 34. In this case, the side hole 42 may be omitted.

In FIG. 2, the needle hub 38 is formed in a hollow shape (cylindrical shape). The constituent material of the needle hub 38 is similar to the constituent material of the catheter hub 22 described above. The needle hub 38 includes an inner needle support portion 44 forming the distal end thereof and a needle hub body 46 extending from the inner needle support portion 44 to the proximal end side.

The proximal end of the inner needle 36 is fixed to the inner needle support portion 44 by using appropriate fixing means such as fusion, adhesion, fitting, or the like. The needle hub body 46 is formed to have a size and shape that can be easily gripped by the user.

As illustrated in FIG. 1, the irradiation unit 14 irradiates the wavelength conversion member 28 of the catheter assembly 12 with the near-infrared light L1. The near-infrared light L1 emitted by the irradiation unit 14 has, for example, a wavelength ranging from 700 nm to 2500 nm.

Next, a blood vessel puncture procedure using the catheter assembly 12 will be described. In the initial state of the catheter assembly 12, the inner needle 36 protrudes in the distal end direction from the distal end opening 23 of the catheter shaft 20 (see FIG. 3).

As illustrated in FIG. 5A, the user punctures the puncture target site 100 with the catheter assembly 12 in the initial state. The puncture target site 100 is, for example, a forearm of a human body. Specifically, for example, the user punctures the subcutaneous tissue 102 of the puncture target site 100. At this time, the irradiation unit 14 irradiates the puncture target site 100 with the near-infrared light L1. Note that the irradiation unit 14 is disposed such that a surface opposite to the surface of the puncture target site 100 to be punctured with the catheter assembly 12 is irradiated with the near-infrared light L1. However, the irradiation unit 14 may be disposed such that the surface of the puncture target site 100 to be punctured with the catheter assembly 12 is irradiated with the near-infrared light L1.

When the puncture target site 100 is punctured with the catheter assembly 12, the catheter distal end 24 is located in the subcutaneous tissue 102. At this time, the near-infrared light L1 is guided to the wavelength conversion member 28 of the catheter distal end 24 through the subcutaneous tissue 102. The wavelength conversion member 28 converts the near-infrared light L1 into the visible light L2. That is, as illustrated in FIG. 5B, the wavelength conversion member 28 emits light in the subcutaneous tissue 102. The user can recognize that the catheter distal end 24 is not located in the blood vessel 104 (the catheter shaft 20 is not secured in the blood vessel) by visually recognizing the visible light L2 generated from the wavelength conversion member 28.

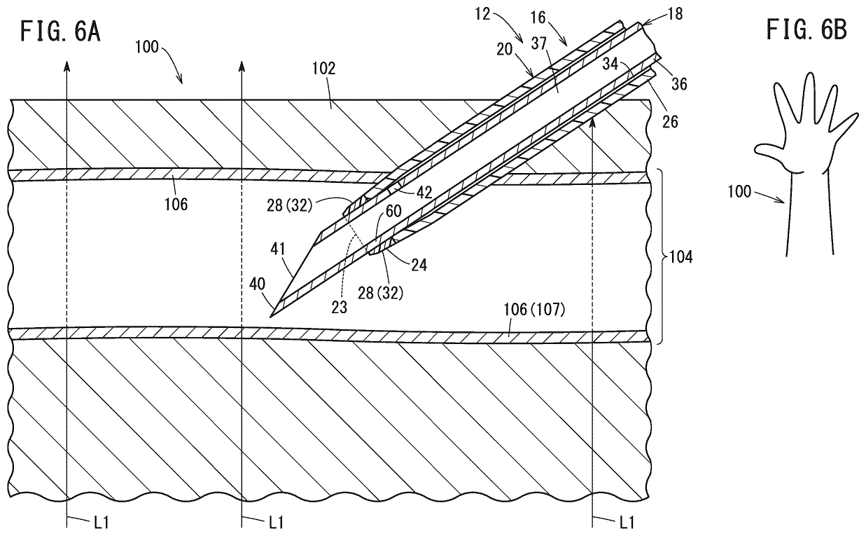

Subsequently, when the catheter assembly 12 is pushed to the puncture target site 100, as illustrated in FIG. 6A, the catheter distal end 24 is located in the blood vessel 104. The near-infrared light L1 is absorbed by red corpuscles (in particular, hemoglobin) of blood flowing in the blood vessel 104. Therefore, the near-infrared light L1 is not emitted to the catheter distal end 24 in the blood vessel 104. That is, as illustrated in FIG. 6B, the visible light L2 is not generated from the wavelength conversion member 28. The user can immediately know that the catheter shaft 20 is secured in the blood vessel by confirming that the visible light L2 generated from the wavelength conversion member 28 disappears.

Furthermore, when the catheter distal end 24 is located in the blood vessel 104, the blood in the blood vessel 104 flows into the lumen 37 of the inner needle 36 from the distal end opening 41 of the inner needle 36. The blood flowing into the lumen 37 of the inner needle 36 is guided to the lumen 25 of the catheter hub 22 via the side hole 42 and the blood introduction passage 34. The user can reconfirm that the catheter distal end 24 is located in the blood vessel 104 by visually recognizing backflow (flashback) of the blood into the lumen 25 of the catheter hub 22.

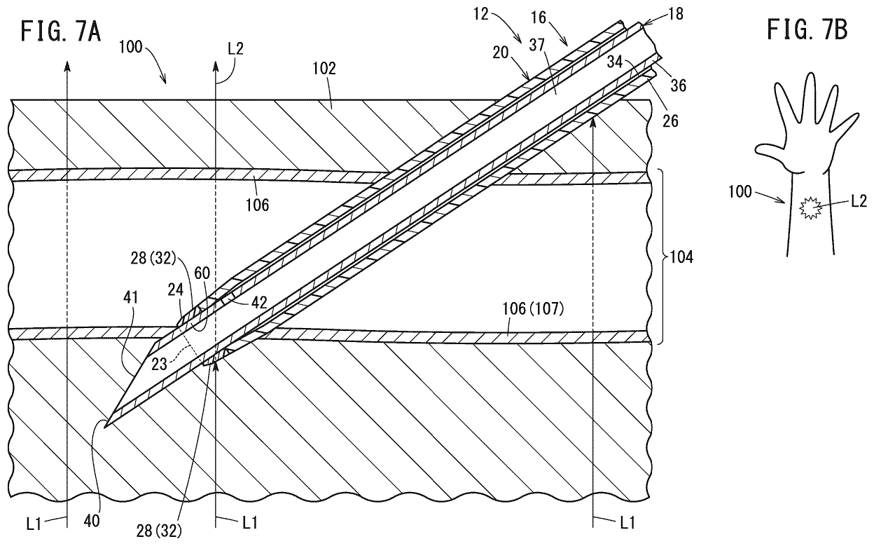

As illustrated in FIG. 7A, in a case in which the inner needle 36 and the catheter shaft 20 penetrate a wall portion 106 (back wall 107) of the blood vessel 104 and the catheter distal end 24 protrudes to the outside of the blood vessel 104 after the catheter shaft 20 is secured in the blood vessel, the near-infrared light L1 is emitted to the catheter distal end 24. Therefore the wavelength conversion member 28 emits light again. That is, as illustrated in FIG. 7B, the wavelength conversion member 28 emits light in the subcutaneous tissue 102 again. The user can know that the catheter distal end 24 protrudes from the back wall 107 of the blood vessel 104 by visually recognizing the re-emission from the wavelength conversion member 28.

In a case in which the catheter distal end 24 comes out of the blood vessel 104 after the catheter shaft 20 is secured in the blood vessel, the wavelength conversion member 28 emits light again. Therefore, the user can know that the catheter distal end 24 comes out of the blood vessel 104 by visually recognizing the re-emission from the wavelength conversion member 28.

After the catheter shaft 20 is secured in the blood vessel, the user removes the inner needle 36 from the catheter shaft 20.

In this case, the present embodiment has the following effects.

By irradiating the wavelength conversion member 28 with the near-infrared light L1 in a state before blood vessel access in which the catheter distal end 24 is located in the subcutaneous tissue 102, the visible light L2 can be generated from the wavelength conversion member 28. Therefore, the user can know that the catheter distal end 24 is located outside the blood vessel 104 by visually recognizing the visible light L2. The near-infrared light L1 is absorbed by the blood flowing in the blood vessel 104. Therefore, when the catheter distal end 24 is located in the blood vessel 104, the near-infrared light L1 is not emitted to the catheter distal end 24. That is, the visible light L2 generated from the wavelength conversion member 28 disappears. Therefore, the user can immediately know that the catheter shaft 20 is secured in the blood vessel by visually recognizing that the visible light L2 generated from the wavelength conversion member 28 disappears.

Furthermore, in a case in which the catheter distal end 24 penetrates the back wall 107 of the blood vessel 104 or the catheter distal end 24 comes out of the blood vessel 104 after the catheter shaft 20 is secured in the blood vessel, the wavelength conversion member 28 emits light again. Therefore, the user can easily know that the catheter distal end 24 is not located in the blood vessel 104 by visually recognizing the re-emission from the wavelength conversion member 28.

Furthermore, by directly visually recognizing the visible light L2 generated from the catheter distal end 24, it is possible to know that the catheter shaft 20 is secured in the blood vessel without using a camera, a monitor, and the like. Therefore, it is possible to prevent the catheter system 10 from becoming large and complicated.

The catheter shaft 20 includes a catheter body 26 that is located on the proximal end side with respect to the catheter distal end 24 and cannot convert the near-infrared light L1 into the visible light L2.

According to this configuration, since the catheter body 26 located outside the blood vessel 104 does not emit light in a state where the catheter distal end 24 is located in the blood vessel 104, it is possible to easily know that the catheter shaft 20 is secured in the blood vessel.

The wavelength conversion member 28 constitutes a wall portion of at least a part of the catheter distal end 24.

According to this configuration, the wavelength conversion member 28 can be easily provided in the catheter distal end 24.

The catheter distal end 24 includes the non-wavelength conversion member 30 that does not include the wavelength conversion material 32.

According to this configuration, the strength of the catheter distal end 24 can be easily adjusted by changing a ratio between the wavelength conversion member 28 and the non-wavelength conversion member 30 in the catheter distal end 24.

The wavelength conversion member 28 is provided only in the catheter distal end 24.

According to this configuration, it can be effectively known that the catheter shaft 20 is secured in the blood vessel.

In the present embodiment, the entire catheter distal end 24 may be configured only by the wavelength conversion member 28. Furthermore, the catheter distal end 24 may be configured by alternately disposing the wavelength conversion members 28 and the non-wavelength conversion members 30 in the axial direction of the catheter shaft 20. In this case, the wavelength conversion member 28 preferably extends annularly in the circumferential direction of the catheter distal end 24. However, the length of the wavelength conversion member 28 in the circumferential direction of the catheter distal end 24 may be less than 360°. Note that in the configuration of the catheter distal end 24, the wavelength conversion member 28 and the non-wavelength conversion member 30 can be appropriately set in the shape, size, number, and the like. Moreover, the configuration of the catheter distal end 24 (configuration in which the wavelength conversion members 28 and the non-wavelength conversion members 30 are alternately disposed in the axial direction of the catheter shaft 20) may be applied over the entire catheter shaft 20.

### First Modification Example

Next, a catheter assembly 12a according to a first modification example will be described. In the present modification example, the same components as those of the above-described catheter assembly 12 are denoted by the same reference numerals, and the detailed description thereof will be omitted. In the present modification example, the same effects are obtained for the same configuration as that of the above-described catheter assembly 12.

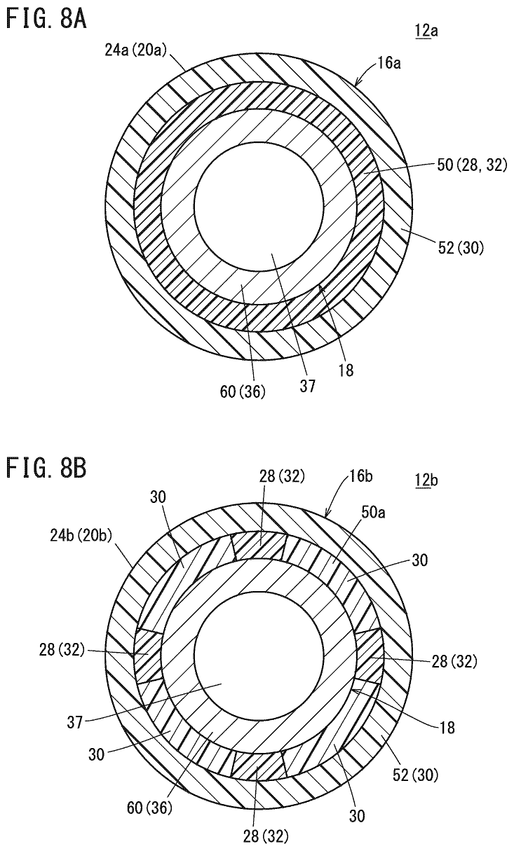

As illustrated in FIG. 8A, the catheter assembly 12a includes a catheter member 16a and the needle member 18. The catheter member 16a includes a catheter shaft 20a. The catheter distal end 24a of the catheter shaft 20a includes an inner layer portion 50 and an outer layer portion 52. The inner layer portion 50 and the outer layer portion 52 are formed in an annular shape. That is, the outer layer portion 52 extends so as to surround the entire outer circumferential surface of the inner layer portion 50. The entire inner layer portion 50 is the wavelength conversion member 28. The entire outer layer portion 52 is the non-wavelength conversion member 30. The wavelength conversion member 28 and the non-wavelength conversion member 30 are adjacent to each other in a radial direction of the catheter shaft 20a.

According to the present modification example, since the wavelength conversion member 28 extends one round in the circumferential direction of the catheter shaft 20a, the appearance of the visible light L2 generated from the wavelength conversion member 28 does not change depending on an angle of viewing the catheter distal end 24a. Therefore, the visible light L2 generated from the wavelength conversion member 28 can be easily seen.

In the present modification example, in the catheter distal end 24a, the entire inner layer portion 50 may be the non-wavelength conversion member 30, and the entire outer layer portion 52 may be the wavelength conversion member 28.

### Second Modification Example

Next, a catheter assembly 12b according to a second modification example will be described. In the present modification example, the same components as those of the above-described catheter assemblies 12 and 12a are denoted by the same reference numerals, and the detailed description thereof will be omitted. In the present modification example, the same effects are obtained for the same configuration as that of the above-described catheter assemblies 12 and 12a.

As illustrated in FIG. 8B, the catheter assembly 12b includes a catheter member 16b and the needle member 18. The catheter member 16b includes a catheter shaft 20b. The catheter distal end 24b of the catheter shaft 20b includes an inner layer portion 50a and the outer layer portion 52. The inner layer portion 50a and the outer layer portion 52 are formed in an annular shape. That is, the outer layer portion 52 extends so as to surround the entire outer circumferential surface of the inner layer portion 50a. The inner layer portion 50a is configured similarly to that of the above-described catheter distal end 24. That is, the inner layer portion 50a includes a plurality of wavelength conversion members 28 and a plurality of non-wavelength conversion members 30. In the present modification example, in the catheter distal end 24b, the entire inner layer portion 50a may be the non-wavelength conversion member 30, and the entire outer layer portion 52 may include a plurality of the wavelength conversion members 28 and a plurality of the non-wavelength conversion members 30.

### Third Modification Example

Next, a catheter assembly 12c according to a third modification example will be described. In the present modification example, the same components as those of the above-described catheter assemblies 12, 12a, and 12b are denoted by the same reference numerals, and the detailed description thereof will be omitted. In the present modification example, the same effects are obtained for the same configuration as that of the above-described catheter assemblies 12, 12a, and 12b.

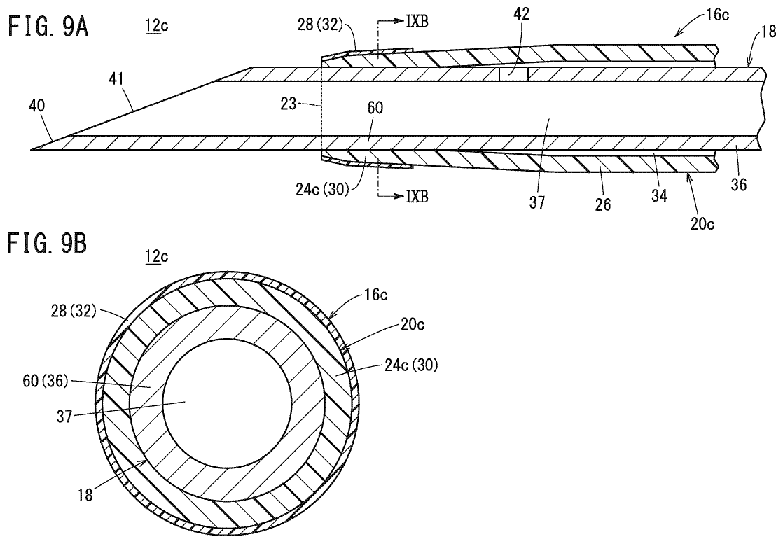

As illustrated in FIGS. 9A and 9B, the catheter assembly 12c includes a catheter member 16c and the needle member 18. The catheter member 16c includes a catheter shaft 20c. The wavelength conversion member 28 is provided on the catheter distal end 24c of the catheter shaft 20c.

The catheter distal end 24c is made of the same material as that of the catheter body 26. That is, the catheter distal end 24c does not include the wavelength conversion material 32. In other words, the catheter distal end 24c is the non-wavelength conversion member 30. The outer circumferential surface of the catheter distal end 24c is coated with the wavelength conversion material 32 to form the wavelength conversion member 28. The wavelength conversion member 28 extends annularly in the circumferential direction of the catheter distal end 24c.

According to the present modification example, since the wavelength conversion member 28 extends circumferentially around the catheter shaft 20c, the appearance of the visible light L2 generated from the wavelength conversion member 28 does not change depending on an angle of viewing the catheter distal end 24c. Therefore, the visible light L2 generated from the wavelength conversion member 28 can be easily seen.

In the present modification example, the wavelength conversion member 28 may be formed by coating not the outer circumferential surface of the catheter distal end 24c but the inner circumferential surface of the catheter distal end 24c with the wavelength conversion material 32. Furthermore, the wavelength conversion member 28 may be formed by coating the outer circumferential surface and inner circumferential surface of the catheter distal end 24c with the wavelength conversion material 32.

### Fourth Modification Example

Next, a catheter assembly 12d according to a fourth modification example will be described. In the present modification example, the same components as those of the above-described catheter assemblies 12 and 12a to 12c are denoted by the same reference numerals, and the detailed description thereof will be omitted. In the present modification example, the same effects are obtained for the same configuration as that of the above-described catheter assemblies 12 and 12a to 12c.

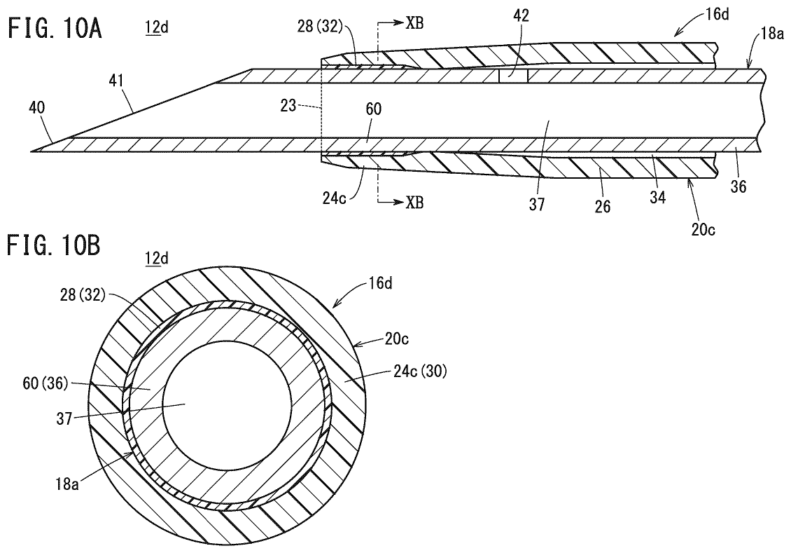

As illustrated in FIGS. 10A and 10B, the catheter assembly 12d includes a catheter member 16d and a needle member 18a. The catheter member 16d is configured similarly to the above-described catheter member 16c except that the wavelength conversion member 28 is not included. The outer circumferential surface of the insertion distal end 60 of the inner needle 36 is coated with the wavelength conversion material 32 to form the wavelength conversion member 28. The wavelength conversion member 28 extends circumferentially around the insertion distal end 60. In the initial state of the catheter assembly 12d, the wavelength conversion member 28 faces the inner circumferential surface of the catheter distal end 24c. The wavelength conversion member 28 is provided only on the insertion distal end 60.

According to the present modification example, since the wavelength conversion member 28 is provided only on the insertion distal end 60, it is possible to effectively know the catheter shaft 20c is secured in the blood vessel.

In the present modification example, the wavelength conversion member 28 may be formed by coating not the outer circumferential surface of the insertion distal end 60 but the inner circumferential surface of the insertion distal end 60 with the wavelength conversion material 32. Furthermore, the wavelength conversion member 28 may be formed by coating the outer circumferential surface and inner circumferential surface of the insertion distal end 60 with the wavelength conversion material 32. Note that in a case in which the inner circumferential surface of the insertion distal end 60 is coated with the wavelength conversion material 32, the insertion distal end 60 is made of a material that transmits the near-infrared light L1.

Note that the present invention is not limited to the above-described disclosure, and various configurations can be adopted without departing from the gist of the present invention. The above-described configurations of the catheter distal ends 24, and 24a to 24c may be applied to all the catheter shafts 20 and 20a to 20c. The catheter assembly according to the present disclosure may be configured by appropriately combining the catheter members 16 and 16a to 16c according to the first to third modification examples and the needle member 18a according to the fourth modification example. The wavelength conversion member 28 may be provided on at least one of the catheter distal ends 24 and 24a to 24c and the insertion distal end 60. Furthermore, the wavelength conversion member 28 may be provided on the entire inner needle 36.

What is claimed is:

1. A catheter assembly comprising:
a tubular catheter shaft; and
an inner needle inserted into a lumen of the catheter shaft, the inner needle comprising an insertion distal end surrounded by a catheter distal end which is a distal end of the catheter shaft in an initial state of the catheter assembly in which the inner needle is inserted into the lumen of the catheter shaft, wherein:
a wavelength conversion member is included in at least one of the catheter distal end or the insertion distal end, the wavelength conversion member comprising a wavelength conversion material that converts near-infrared light into visible light.

2. The catheter assembly according to claim 1, wherein:
the wavelength conversion member is included in the catheter distal end, and
the catheter shaft comprises a catheter body that is located on a proximal end side with respect to the catheter distal end and is not capable of converting the near-infrared light into the visible light.

3. The catheter assembly according to claim 2, wherein:
the wavelength conversion member constitutes a wall portion of at least a part of the catheter distal end.

4. The catheter assembly according to claim 2, wherein:
the catheter distal end comprises a non-wavelength conversion member that does not include the wavelength conversion material.

5. The catheter assembly according to claim 4, wherein:
the wavelength conversion member and the non-wavelength conversion member are adjacent to each other in a circumferential direction or a radial direction of the catheter shaft.

6. The catheter assembly according to claim 2, wherein:
the wavelength conversion member is coated on at least one of an outer circumferential surface or an inner circumferential surface of the catheter distal end.

7. The catheter assembly according to claim 1, wherein:
the wavelength conversion material converts the near-infrared light having a wavelength of more than 700 nm into the visible light having a wavelength ranging from 360 nm to 700 nm.

8. The catheter assembly according to claim 1, wherein:
the wavelength conversion member is included only in the catheter distal end or only on the insertion distal end.

9. A catheter system comprising:
a catheter assembly comprising:
a tubular catheter shaft, and
an inner needle inserted into a lumen of the catheter shaft, the inner needle comprising an insertion distal end surrounded by a catheter distal end which is a distal end of the catheter shaft in an initial state of the catheter assembly in which the inner needle is inserted into the lumen of the catheter shaft, wherein:
a wavelength conversion member is included in at least one of the catheter distal end or the insertion distal end, the wavelength conversion member comprising a wavelength conversion material that converts near-infrared light into visible light; and
an irradiation unit for emitting the near-infrared light.

10. A method for puncturing a blood vessel, the method comprising:
providing a catheter assembly comprising:
a tubular catheter shaft, and
an inner needle inserted into a lumen of the catheter shaft, the inner needle comprising an insertion distal end surrounded by a catheter distal end which is a distal end of the catheter shaft in an initial state of the catheter assembly in which the inner needle is inserted into the lumen of the catheter shaft, wherein:
a wavelength conversion member is included in at least one of the catheter distal end or the insertion distal end, the wavelength conversion member comprising a wavelength conversion material that converts near-infrared light into visible light;
puncturing a target site with the catheter assembly in the initial state;
irradiating the target site with the near-infrared light, such that the wavelength conversion member emits the visible light; and
determining that the catheter is not located in the blood vessel by visually recognizing the visible light generated from the wavelength conversion member; and
determining that the catheter distal end is located in the blood vessel by visually recognizing that the visible light generated from the wavelength conversion member disappears.
